(19) United States
(12) Patent Application Publication
(10) Pub. No.: US 2017/0239448 A1
(43) Pub. Date: Aug. 24, 2017

(54) ANCHORING GUIDEWIRE

(71) Applicant: Boston Scientific Scimed, Inc., Maple Grove, MN (US)

(72) Inventors: Hong Cao, Maple Grove, MN (US); Sean Fleury, Minneapolis, MN (US)

(21) Appl. No.: 15/435,848

(22) Filed: Feb. 17, 2017

Related U.S. Application Data
(60) Provisional application No. 62/296,878, filed on Feb. 18, 2016.

(51) Int. Cl. A61M 25/04; A61B 1/267; A61B 1/018; A61B 10/04; A61M 25/09; A61M 25/01

(52) U.S. Cl. CPC A61M 25/04; A61M 25/09; A61M 25/0133; A61B 1/018; A61B 10/04; A61B 1/2676; A61M 2025/09175; A61M 2025/09075; A61M 2025/09066

(57) ABSTRACT

The present disclosure relates to the field of endoscopy. Specifically, the present disclosure relates to systems and methods for anchoring guidewires within body passageways to provide efficient and accurate positioning and/or exchange of medical instruments to a target location. More specifically, the present disclosure relates to an anchoring guidewire that includes a self-expanding coil for precise positioning and/or exchange of a biopsy tool within a bronchial passageway.

### CROSS-REFERENCE TO RELATED APPLICATIONS

This application claims the benefit of priority to U.S. Provisional Application Ser. No. 62/296,878, filed Feb. 18, 2016, the disclosure of which is herein incorporated by reference in its entirety.

### FIELD

The present disclosure relates to the field of endoscopy. Specifically, the present disclosure relates to systems and methods for anchoring guidewires within body passageways to facilitate accurate positioning and/or exchange of medical instruments to a target location. In particular, the present disclosure relates to an anchoring guidewire that includes a self-expanding coil for precise positioning and/or exchange of medical instruments within a bronchial passageway.

### BACKGROUND

Guidewires are commonly used in surgical procedures to facilitate smooth tracking and rapid exchange of medical instruments through the tortuous anatomy of the patient. Conventional guidewires, such as those including a tapered flexible tip, allow target locations to be accessed, but do not prevent the guidewire from moving once properly positioned. The propensity for such guidewires to move during a medical procedure, especially when medical instruments are exchanged over the guidewire, tends to prevent the accurate delivery of subsequent instruments to the original/target location. This is especially problematic for endoscopy procedures performed within narrow body lumens, such as the bronchial passageways, which are only accessible using small diameter endoscopes that include a single working channel. These space constraints may require multiple exchanges of medical instruments over the guidewire through the single working channel of the endoscope during the endoscopy. Even slight movement of the guidewire during the exchange of medical instruments may prevent the subsequently introduced medical instrument from accessing the intended target tissue. The inability to ensure and maintain accurate positioning of exchanged medical instruments may result in a variety of negative outcomes, including, prolonged, failed or improperly performed procedures.

There is an ongoing need, particularly in the endoscopy field, for an anchoring guidewire that maintains a fixed position within a passageway during medical instrument exchange, and which is easily retractable once the procedure is completed.

### SUMMARY

The present disclosure, in its various aspects, meets an ongoing need in the medical field, such as the field of endoscopy, for a guidewire that includes a self-expanding distal retention member to anchor the guidewire within body passageways so that medical instruments may be efficiently and accurately exchanged and delivered to a target location.

In one aspect, the present disclosure provides an anchoring guidewire, comprising: an elongate core wire having a proximal portion and a distal portion; a retention member disposed at a distal end of the elongate core wire; and an extendable-retractable sheath slidably disposed about at least a portion of the elongate core wire and the retention member; wherein the retention member is in a first configuration with a first outer diameter when disposed inside the extendable-retractable sheath and a second configuration with a second outer diameter when disposed outside the extendable-retractable sheath; and wherein the second outer diameter is larger than the first outer diameter. The retention member may include a helically-biased wire formed in the shape of a coil. The retention member is configured to contact a wall of a body lumen when in the second configuration. The retention member may be formed from a distal winding of the elongate core wire. Alternatively, the retention member may be attached to the distal end of the elongate core wire by a weld, solder, adhesive, glue, resin or the like. The retention member may include a shape memory material selected from the group consisting of platinum, tungsten, titanium, stainless steel, nickel, alloys thereof and the like. The elongate core wire may include a metal selected from the group consisting of platinum, tungsten, titanium, stainless steel, nickel, alloys thereof and the like. The extendable-retractable sheath may include a polymer selected from the group consisting of acrylate-based polymers, polyurethane-based polymers, polynorbornene-based polymer, polylactide-based polymers and the like. The elongate core wire may be flexible. For example, the elongate core wire may include various levels or degrees of flexibility along its length to facilitate navigation within and through body passageways. The distal portion of the elongate core wire may be steerable by, for example, manipulating the proximal portion of the elongate core wire. Retracting the extendable-retractable sheath in a proximal direction relative to the distal end of the elongate core wire may release the retention member from within the extendable-retractable sheath, while advancing the extendable-retractable sheath in a distal direction relative to the distal end of the elongate core wire may return the retention member into the extendable-retractable sheath. The elongate core wire; retention member; and extendable-retractable sheath may be configured to be slidably disposed within a working channel of a medical device (e.g., catheter, bronchoscope, ureteroscope, duodenoscope, colonoscope, arthroscope, cystoscope, hysteroscope, stent, ultrasound device and the like), or a working channel of a medical instrument (e.g., biopsy tool, grasping element, cutting element and the like).

In another aspect, the present disclosure provides a system, comprising: a medical device comprising an elongate body having a proximal end, a distal end and a working channel extending therebetween; an anchoring guidewire slidably disposed within the working channel of the medical device, the anchoring guidewire comprising an elongate core wire having a proximal portion and a distal portion; a retention member disposed at a distal end of the elongate core wire; and an extendable-retractable sheath slidably disposed about at least a portion of the elongate core wire and the retention member; wherein the retention member is in a first configuration with a first outer diameter when disposed inside the extendable-retractable sheath, and a second configuration with a second outer diameter when disposed outside the extendable-retractable sheath; and wherein the second outer diameter is larger than the first outer diameter. A medical instrument may be slidably disposed about the anchoring guidewire within the working channel of the medical device. The medical device may include a catheter, bronchoscope, ureteroscope, duodenoscope, colonoscope, arthroscope, cystoscope, hysteroscope, stent or an ultrasound device. The medical instrument may include a biopsy tool, grasping element or a cutting element.

In yet another aspect, the present disclosure provides a method, comprising: inserting an anchoring guidewire into a body lumen, the anchoring guidewire comprising an elongate core wire; a retention member disposed at a distal end of the elongate core wire; and a sheath slidably disposed about at least a portion of the elongate core wire; positioning the distal end of the elongate core wire proximate to a target tissue; and sliding the sheath relative to the distal end of the elongate core wire such that the retention member expands to a form that has a diameter greater than a diameter of the wire and sheath. The retention member may expand to the form of a helically-biased coil. The retention member may contact a body lumen wall when in the expanded form, thereby securing the anchoring guidewire within the body lumen. The method may further include advancing a tool over the anchoring guidewire to the target tissue site.

### BRIEF DESCRIPTION OF THE DRAWINGS

Non-limiting examples of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of skill in the art to understand the disclosure. In the figures:

FIGS. 1A-1B illustrate the distal end of an anchoring guidewire in a constrained delivery (FIG. 1A) and unconstrained/deployed (FIG. 1B) configurations, according to an embodiment of the present disclosure.

FIG. 2 illustrates a bronchoscope and anchoring guidewire positioned within a bronchial passageway of a patient, according to an embodiment of the present disclosure.

FIGS. 3A-3E illustrate the various steps involved in exchanging a medical instrument over an anchoring guidewire, according to an embodiment of the present disclosure.

FIGS. 4A-4B illustrate alternative anchoring guidewire configurations, according to other embodiments of the present disclosure.

It is noted that the drawings are intended to depict only typical or exemplary embodiments of the disclosure. Accordingly, the drawings should not be considered as limiting the scope of the disclosure. The disclosure will now be described in greater detail with reference to the accompanying drawings.

### DETAILED DESCRIPTION

Before the present disclosure is described in further detail, it is to be understood that the disclosure is not limited to the particular embodiments described, as such may vary. It is also to be understood that the terminology used herein is for the purpose of describing particular embodiments only, and is not intended to be limiting beyond the scope of the appended claims. Unless defined otherwise, all technical terms used herein have the same meaning as commonly understood by one of ordinary skill in the art to which the disclosure belongs. Finally, although embodiments of the present disclosure are described with specific reference to anchoring guidewires for use within narrow bronchial passageways, it should be appreciated that such anchoring guidewires may be used in a variety of body lumens, including, for example, the heart, vascular system, circulatory system, gastrointestinal (GI) tract, stomach, esophagus, urogenital system and the like.

As used herein, the term “distal” refers to the end farthest away from a medical professional when introducing a device into a patient, while the term “proximal” refers to the end closest to the medical professional when introducing a device into a patient.

As used herein, the term “guidewire” refers to an elongated core wire device configured to readily advance through body passageways to a location at which a medical procedure or treatment is to take place. A variety of medical tools (e.g., navigation catheters, biopsy tools, grasping or cutting elements) may then be advanced over the inserted guidewire to follow the pathway defined by the guidewire.

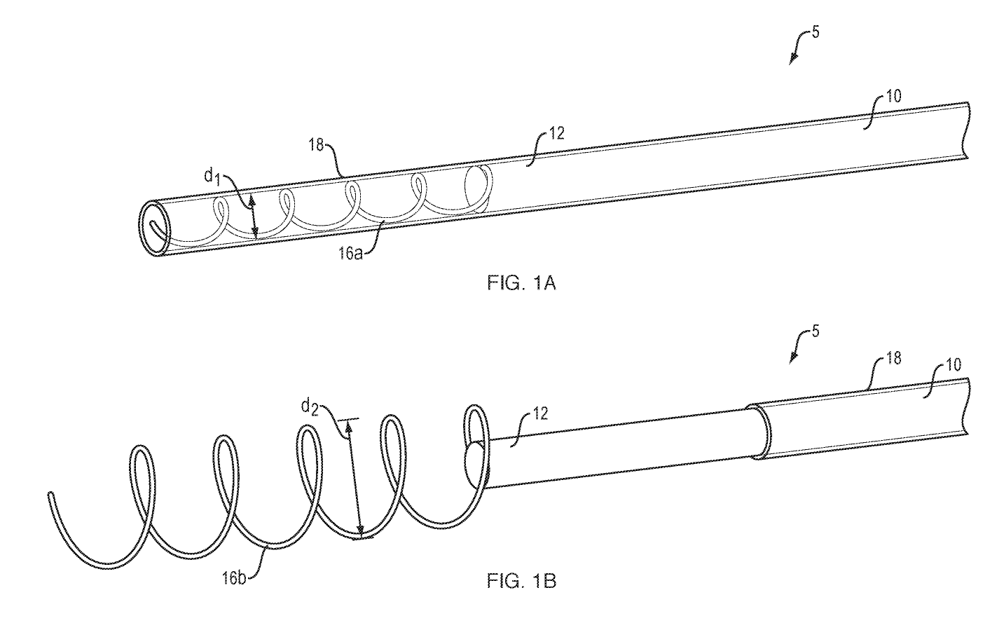

In one embodiment, the present disclosure provides systems and methods for anchoring guidewires within body passageways to provide efficient and accurate positioning and exchange of medical instruments to a target location. As illustrated in FIG. 1A, the anchoring guidewire 5 of the present disclosure may include an elongate core wire 10 comprising a distal portion 12 and a proximal portion (not shown). A retention member 16a may be disposed at a distal end of the elongate core wire 10. An extendable-retractable sheath 18 may be slidably disposed about at least a portion of the elongate core wire 10 and the retention member 16a.

The elongate core wire 10 may be made from a variety of kink resistant materials, including, for example, platinum, tungsten, titanium, stainless steel, nickel and nickel-titanium alloys (e.g., nitinol). Nitinol may be preferred due to its superelasticity characteristics which resist kinking and permit the elongate core wire 10 to bend around sharp radius turns without becoming permanently deformed. Nitinol also has greater torquability than conventionally used materials, thereby increasing the steerability of the elongate core wire 10. As will be understood by those in the art, the medical professional may advance the guidewire through body passageways by applying pressure/torque to a proximal end of the elongate core wire 10. These torque forces are transmitted along the length of the elongate core wire 10 to orient/reorient the flexible/bendable distal portion 12 within and through the body passageway. In one embodiment, the elongate core wire 10 may include various levels of flexibility along its length to improve steerability. For example, the diameter of the core wire may be varied along its length to provide regions of increased (e.g., smaller diameter) and decreased (e.g., larger diameter) flexibility. In addition, or alternatively, the core wire may include discrete segments of different kink resistant materials with varying flexibilities.

The elongate core wire 10 of the present disclosure may be provided in a wide variety of lengths corresponding to the length of the medical devices with which they are used, as well as the anatomical target site within the patient. In general, the elongate core wire 10 is necessarily longer than the medical device with which it is intended to be used so that it may be manipulated from its proximal end while the distal end projects beyond the distal end of the medical device. By way of non-limiting example, the length of the elongate core wire 10 may be from about 80 centimeters to about 400 centimeters; and more preferably from about 100 centimeters to about 175 centimeters. The elongate core wire 10 may also include an outer diameter from about 0.5 millimeters to about 2.0 millimeters; and more preferably from about 1.0 millimeters to about 1.5 millimeters.

In one embodiment, the retention member may be attached to, and extend distally from, a distal end of the elongate core wire 10. As will be understood by those in the art, the retention member may be attached to the elongate core wire 10 by one of a weld, solder, braze, adhesive, glue or resin. Alternatively, the retention member may be formed as a continuous winding of the distal portion 12 of the elongate core wire 10. In either embodiment, the retention member may extend approximately 20 millimeters or less (e.g., 15 millimeters or less; 10 millimeters or less; 5 millimeters or less) beyond the distal end of the elongate core wire 10 to provide beneficial purchase with the tissues of the body lumen wall. Although the retention member of FIGS. 1A-1B includes approximately four complete windings, it should be appreciated that the number of windings may be greater than four (i.e., 5 or more windings) or less than four (i.e., 3 or fewer windings).

The retention member may have a first (i.e., constrained or unexpanded) configuration 16a with a first outer diameter d₁ when disposed inside the extendable-retractable sheath 18 (FIG. 1A), and a second (i.e., unconstrained or expanded) configuration 16b with a second outer diameter d₂ greater than the first outer diameter d₁ when disposed outside the extendable-retractable sheath 18 (FIG. 1B). For example, in one embodiment the retention member may have a first outer diameter d₁ of approximately 0.5 millimeters to approximately 2.0 millimeters when in the first configuration 16a, and a second outer diameter d₂ approximately three times greater than the first outer diameter d₁ (e.g., approximately 1.5 millimeters to approximately 6.0 millimeters) when in the second configuration 16b. It should be appreciated that such first and second outer diameters are provided as non-limiting examples of potential configurations of the retention member.

The retention member may be formed from a variety of resilient biologically compatible materials, including metals and metal alloys, such as platinum, tungsten, titanium, stainless steel, nickel and nickel-titanium alloys (e.g., nitinol); polymers such as acrylate-based polymers, polyurethane-based polymers, polynorbornene-based polymers, and polylactide-based polymers and combinations thereof. These materials may be formed into a wire having an outer diameter similar to that of the elongate core wire, e.g., from about 0.5 millimeters to about 2.0 millimeters; and more preferably from about 1.0 millimeters to about 1.5 millimeters.

The self-expanding and resilient nature of these materials allows the retention member to expand according to the diameter of any passageway within which it is deployed, thereby positioning the anchoring guidewire 5 within larger or smaller body passageways. Finally, although the retention member of FIGS. 1A-1B is shown in the shape of helical coil, it should be appreciated that a variety of self-expanding and/or collapsible-expandable retention members (e.g., spherical, oblong or elongate frameworks; extendable-retractable fingers; inflatable balloons and the like) may be disposed at the distal end of the elongate core wire 10 to contact and anchor against the body lumen wall.

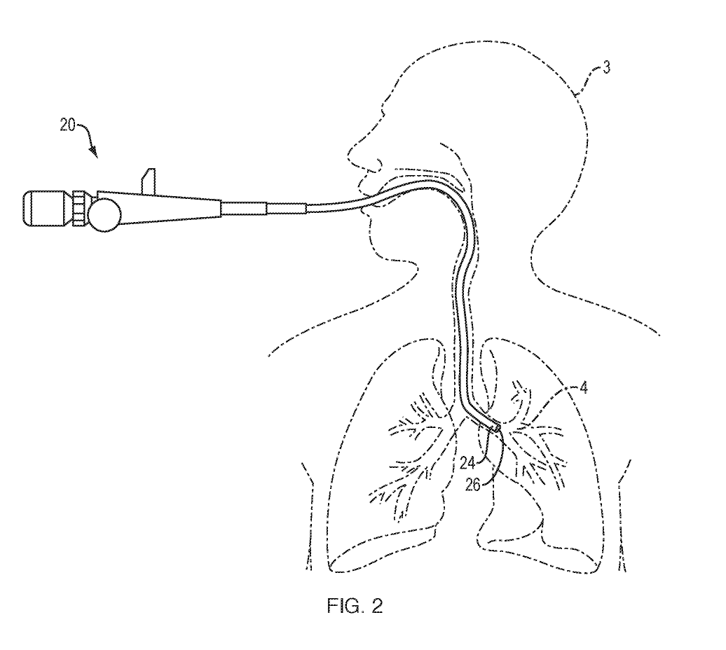

The extendable-retractable sheath 18 may be slidably disposed about, and extend longitudinally along, the length of the elongate core wire 10. The retention member may be constrained in a first configuration 16a when disposed within the extendable-retractable sheath 18 (FIG. 1A) to allow the anchoring guidewire 5 to pass through the working channel of a medical device (FIG. 2). The extendable-retractable sheath 18 may be retracted in a proximal direction relative to the distal end of the elongate core wire 10 such that the retention member moves from the first configuration 16a (FIG. 1A) to the second configuration 16b (FIG. 1B). The extendable-retractable sheath 18 may likewise be advanced in a distal direction relative to the distal end of the elongate core wire 10 such that the retention member returns to the first configuration 16a (FIG. 1A) inside the extendable-retractable sheath 18. Alternatively, the extendable-retractable sheath 18 may remain stationary and the elongate core wire 10 advanced in a distal direction and retracted in a proximal direction to push and pull the retention member back and forth into the extendable-retractable sheath 18. As discussed above, the self-expanding and resilient nature of the retention member allows successive helical windings to individually collapse as they are drawn back inside the extendable-retractable sheath 18, thereby returning the retention member to the first configuration 16a.

The extendable-retractable sheath 18 may be include a wall thickness of approximately 4.0 millimeters or less (e.g., 3.0 millimeters or less; 2.0 millimeters or less; 1.0 millimeters or less; 0.5 millimeters or less; 0.05 millimeters or less) formed from a variety of flexible/bendable polymers comprising, for example, nylon (e.g., such as nylon 12, nylon 11, nylon 6/12, nylon 6, nylon 66), polyesters (e.g., polyethylene terephthalate (PET), polybutylene terephthalate (PBT), polyethylene naphthalate (PEN), polytrimethylene terephthalate (PTT); polyethers; polyurethanes; polyvinyls; polyacrylics; fluoropolymers; copolymers and block copolymers thereof, such as block copolymers of polyether and polyamide (e.g., PEBAX®); and mixtures thereof. The extendable-retractable sheath 18 may further include a coating (e.g., lubricous hydrophobic and/or hydrophilic coating) on an exterior and/or interior surface thereof to facilitate low friction movement. For example, an exterior lubricious coating may facilitate low friction movement along an outer surface of the extendable-retractable sheath during positioning and/or exchange of the medical device. Similarly, an interior lubricious coating may facilitate low friction movement of the elongate core wire through the extendable-retractable sheath as the retention member moves between the first and second configurations.

Referring to FIG. 2, the systems and methods of the present disclosure may include a medical device 20 (e.g., bronchoscope) that includes an elongate body comprising a proximal end, a distal end 24 and a working channel 26 extending therebetween. It should be appreciated that the medical devices of the present disclosure are not limited to bronchoscopes, and may include a variety of medical devices for accessing body passageways, including, for example, catheters, ureteroscopes, duodenoscopes, colonoscopes, arthroscopes, cystoscopes, hysteroscopes, stents, ultrasound devices and the like. As shown, a medical device 20 may be inserted into and advanced through the trachea of a patient 3 such that the distal end 24 is positioned adjacent to a target tissue within a bronchial passageway.

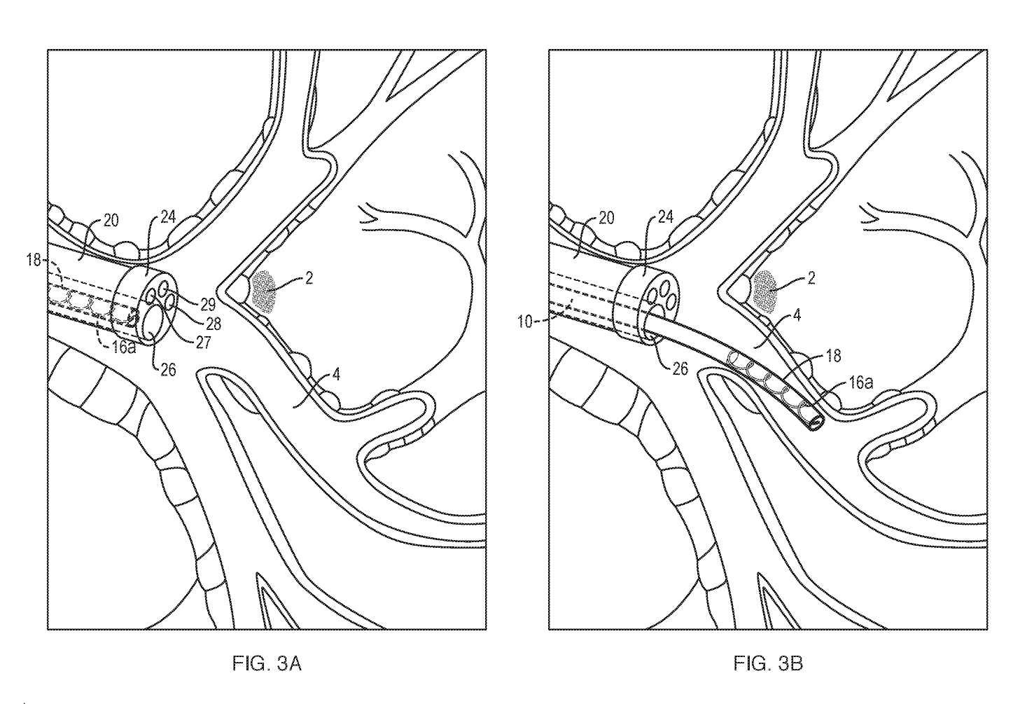

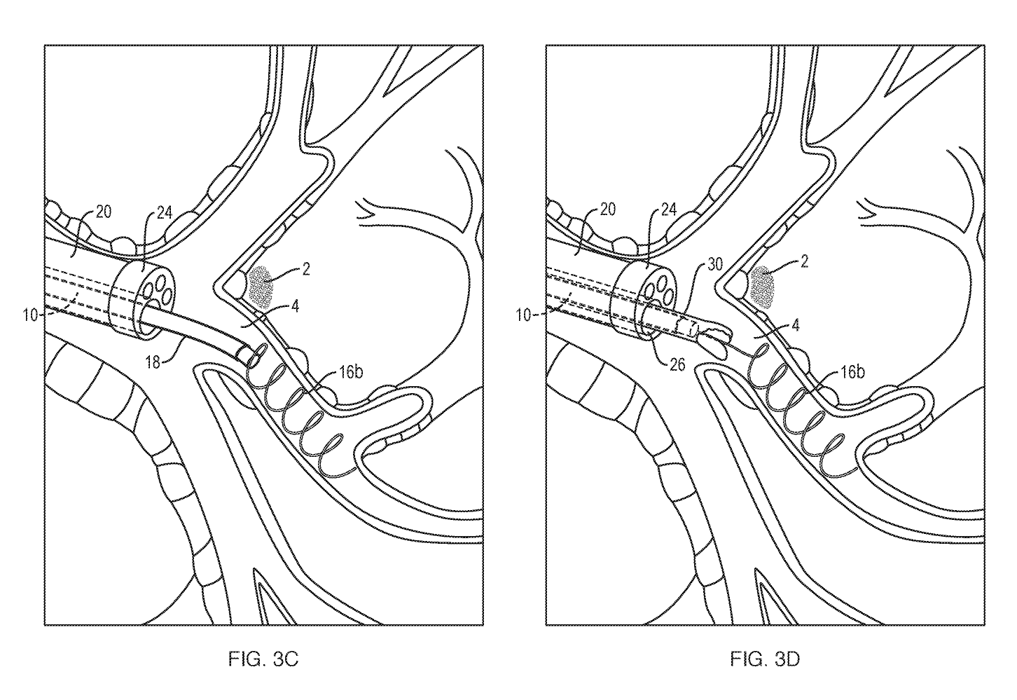

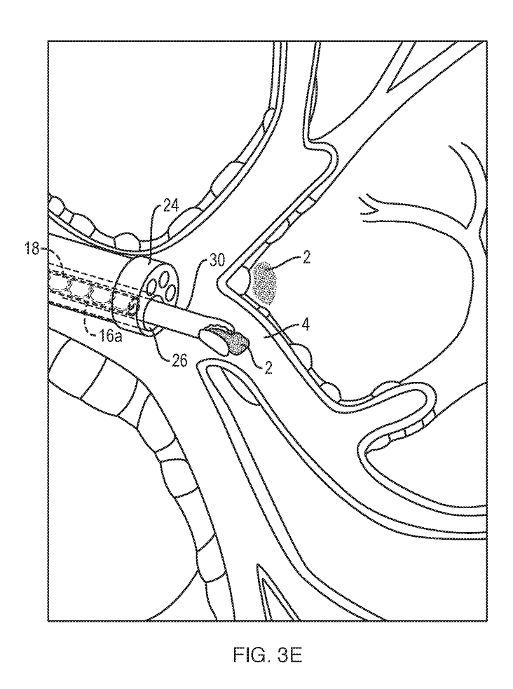

Referring to FIG. 3A, the distal end 24 of the medical device 20 may include a working channel 26, light 27, camera 28 and sensor 29 (e.g., embedded electromagnetic sensor or radiopaque material) to allow the medical professional to position the distal end 24 at the proper location within the bronchial passageway 4 and identify a target tissue 2. In one embodiment, the elongate core wire (not shown), retention member 16a and extendable-retractable sheath 18 may be disposed within the working channel 26 as the medical device 20 is positioned within the bronchial passageway 4 on one side of the target tissue 2. Alternatively, the elongate core wire, retention member 16a and extendable-retractable sheath 18 may be advanced through the working channel 26 after the distal end 24 of the medical device has been properly positioned within the patient.

The elongate core wire 10 may then be advanced through the working channel 26 beyond the distal end 24 of the medical device 20 and further into the bronchial passageway 4 such that the retention member 16a and extendable-retractable sheath 18 are positioned on the other (i.e., opposite) side of the target tissue 2 (FIG. 3B). The extendable-retractable sheath 18 may then be retracted in a proximal direction relative to the elongate core wire 10 such that the retention member moves to the second configuration 16b to contact the wall of the bronchial passageway 4, thereby positioning the elongate core wire 10 (FIG. 3C). A medical instrument 30 (e.g., a biopsy tool etc.) may then be advanced over the anchored elongate core wire 10 through the working channel 26 of the medical device 20 to the target tissue 2 (FIG. 3D). Once the medical instrument 30 is properly positioned adjacent to the target tissue 2, the retention member and extendable-retractable sheath 18 may be retracted into the working channel 26 of the medical device 20 such that movement of the medical instrument 30 within the bronchial passageway 4 is not hindered (FIG. 3E). For example, the medical instrument 30 may include a grasping or cutting element that allows the medical professional to obtain a sample of the target tissue 2 that may be retrieved through the working channel 26 for subsequent analysis.

In one embodiment, the elongate core wire 10 and extendable-retractable sheath 18 may be retracted in a proximal direction relative to the medical device 20 such that the retention member is pulled into the working channel 26, thereby returning to the first configuration 16a. In another embodiment, the elongate core wire 10 may be retracted in a proximal direction relative to the extendable-retractable sheath 18 such that the retention member returns to the first configuration 16a within the extendable-retractable sheath 18 prior to being retracted into the working channel 26 of the medical device 20. In yet another embodiment, the extendable-retractable sheath may be advanced in a distal direction relative to the elongate core wire 10 such that the retention member returns to the first configuration 16a within the extendable-retractable sheath 18 prior to being retracted into the working channel 26 of the medical device 20.

If the medical procedure requires the use of a second medical instrument, the retention member may be re-deployed on the opposite side of the target tissue as outlined above. Once the retention member 16a is properly anchored against the wall of the bronchial passageway 4, the medical instrument 30 may be advanced over the elongate core wire 10 through the working channel 26 of the medical device 20 to the site of the target tissue 2 without disrupting or otherwise moving the previously-established anchoring point. A second medical instrument may then be positioned at the target tissue site as outlined above.

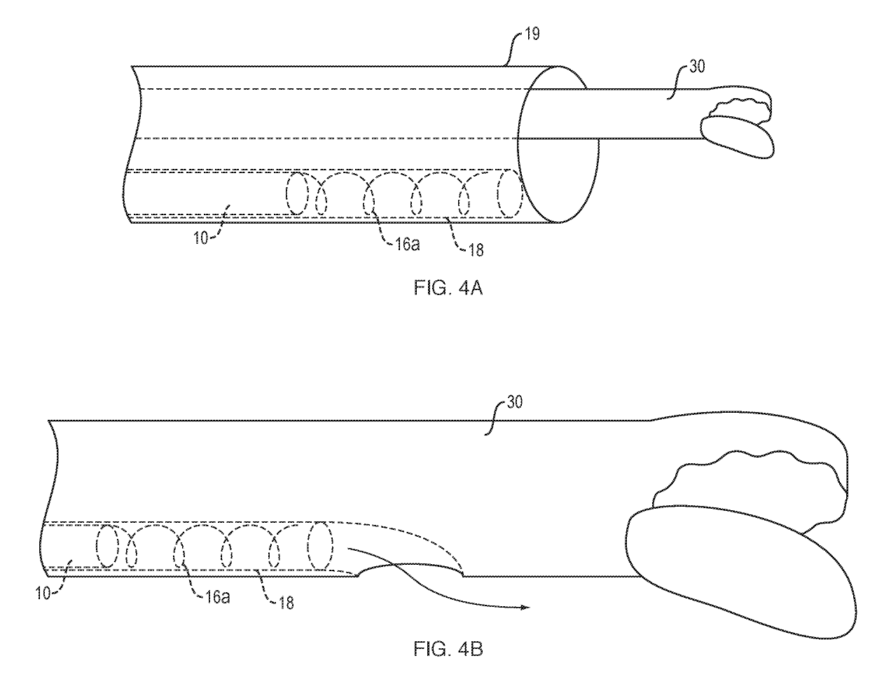

In another embodiment, the elongate core wire 10, retention member 16a and extendable-retractable sheath 18 of the anchoring guidewire may be housed within a sheath 19 (FIG. 4A). Rather than passing over the anchoring guidewire, the medical instrument 30 may be slidably disposed within the sheath 19 alongside the anchoring guidewire. The sheath 19 may be advanced through the lumen of the medical device to the target tissue as described above. Once the medical device is properly positioned, the elongate core wire 10 may be advanced through the bronchial passageway and anchored as discussed above. The medical instrument 30 may then be deployed and maneuvered within the bronchial passageway without hindrance by the elongate core wire 10 or retention member 16a. In another embodiment, rather than advancing the full length of the medical instrument 30 over the anchoring guidewire (e.g., FIGS. 3D and 3E), a distal portion of the medical instrument 30 may be offset from the anchoring guidewire (FIG. 4B). The elongate core wire 10, retention member 16a and extendable-retractable sheath 18 may be advanced through the bronchial passageway and anchored beyond the distal end of the medical instrument, thereby allowing the offset distal portion of the medical instrument to be deployed and maneuvered while the elongate core wire 10 remains secured/anchored against the wall of the bronchial passageway.

Finally, although the embodiments of the present disclosure have been described in use with a medical device, it should be appreciated that the anchoring guidewire of the present disclosure may be positioned within the patient in the absence of an accompanying medical device 20. For example, the anchoring guidewire may be introduced into the patient through a working channel of the medical instrument itself. Alternatively, the anchoring guidewire may be introduced into the patient by itself. Once the retention member is properly anchored, one or more medical instruments may be deployed and/or exchanged over the elongate core wire to the site of the target tissue as discussed above.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. An anchoring guidewire, comprising:
an elongate core wire having a proximal portion and a distal portion;
a retention member disposed at a distal end of the elongate core wire; and
an extendable-retractable sheath slidably disposed about at least a portion of the elongate core wire and the retention member;
wherein the retention member is in a first configuration with a first outer diameter when disposed inside the extendable-retractable sheath and a second configuration with a second outer diameter when disposed outside the extendable-retractable sheath; and
wherein the second outer diameter is larger than the first outer diameter.

2. The anchoring guidewire of claim 1, wherein the retention member includes a wire formed in a shape of a coil.

3. The anchoring guidewire of claim 1, wherein the retention member is configured to contact a wall of a body lumen when in the second configuration.

4. The anchoring guidewire of claim 1, wherein the retention member comprises a distal winding of the elongate core wire.

5. The anchoring guidewire of claim 1, wherein the retention member is attached to the distal end of the elongate core wire by one of a weld, solder, adhesive, glue or resin.

6. The anchoring guidewire of claim 1, wherein the elongate core wire comprises a metal selected from the group consisting of platinum, tungsten, titanium, stainless steel, nickel and alloys thereof.

7. The anchoring guidewire of claim 1, wherein the retention member comprises a shape memory material selected from the group consisting of platinum, tungsten, titanium, stainless steel, nickel and alloys thereof.

8. The anchoring guidewire of claim 1, wherein extendable-retractable sheath comprises a polymer selected from the group consisting of acrylate-based polymers, polyurethane-based polymers, polynorbornene-based polymer and polylactide-based polymers.

9. The anchoring guidewire of claim 1, wherein a distal portion of the elongate core wire is steerable.

10. The anchoring guidewire of claim 9, wherein the distal portion of the elongate core wire is steerable by manipulating the proximal portion of the elongate core wire.

11. The anchoring guidewire of claim 1, wherein retracting the extendable-retractable sheath in a proximal direction relative to the distal end of the elongate core wire releases the retention member from within the extendable-retractable sheath.

12. The anchoring guidewire of claim 1, wherein advancing the extendable-retractable sheath in a distal direction relative to the distal end of the elongate core wire returns the retention member into the extendable-retractable sheath.

13. A system, comprising:
a medical device comprising
an elongate body having a proximal end, a distal end and a working channel extending therebetween;
an anchoring guidewire slidably disposed within the working channel of the medical device, the anchoring guidewire comprising
an elongate core wire having a proximal portion and a distal portion;
a retention member disposed at a distal end of the elongate core wire; and
an extendable-retractable sheath slidably disposed about at least a portion of the elongate core wire and the retention member;
wherein the retention member is in a first configuration with a first outer diameter when disposed inside the extendable-retractable sheath, and a second configuration with a second outer diameter when disposed outside the extendable-retractable sheath; and
wherein the second outer diameter is larger than the first outer diameter.

14. The system of claim 13, further comprising a medical instrument slidably disposed about the anchoring guidewire within the working channel of the medical device.

15. The system of claim 13, wherein the medical device is an endoscope.

16. The system of claim 14, wherein the medical instrument is a biopsy tool.

17. A method, comprising:
inserting an anchoring guidewire into a body lumen, the anchoring guidewire comprising
an elongate core wire;
a retention member disposed at a distal end of the elongate core wire; and
a sheath slidably disposed about at least a portion of the elongate core wire;
positioning the distal end of the elongate core wire proximate to a target tissue; and
sliding the sheath relative to the distal end of the elongate core wire such that the retention member expands to a form that has a diameter greater than a diameter of the wire and sheath.

18. The method of claim 17, wherein the retention member is a helically-biased coil.

19. The method of claim 17, wherein the retention member contacts a body lumen wall when in the expanded form, thereby securing the anchoring guidewire within the body lumen.

20. The method of claim 17, further comprising advancing a tool over the anchoring guidewire to the target tissue.
